(19) United States
(12) Patent Application Publication
(10) Pub. No.: US 2020/0216314 A1
(43) Pub. Date: Jul. 9, 2020

(54) CHLORINE DIOXIDE GAS GENERATING METHOD, LIQUID COMPOSITION, GEL COMPOSITION, AND CHLORINE DIOXIDE GAS GENERATING KIT

(71) Applicant: CLO2 LAB INC., Nishinomiya-shi, Hyogo (JP)

(72) Inventors: Koji ABE, Nishinomiya-shi, Hyogo (JP); Tsukasa ABE, Nishinomiya-shi, Hyogo (JP)

(21) Appl. No.: 16/645,477

(22) PCT Filed: Aug. 28, 2018
(86) PCT No.: PCT/JP2018/031724

§ 371 Date: Mar. 8, 2020

(30) Foreign Application Priority Data
Sep. 20, 2017 (JP) 2017-180688

(51) Int. Cl. C01B 11/02; A61L 2/20; A61L 9/04; A01N 25/04; A01N 59/00

(52) U.S. Cl. CPC C01B 11/024; A61L 2/20; A61L 9/046; A61L 2209/21; A01N 25/04; A01N 59/00; A61L 9/048

(57) ABSTRACT

Chlorine dioxide gas is generated at a stable concentration from a liquid composition. The composition is obtained by mixing an aqueous chlorite solution, an activator that immediately adjusts a pH of the aqueous chlorite solution, thereby causing the aqueous chlorite solution to generate chlorine dioxide gas, and an activation inhibitor that slowly mitigates an action of the activator.

### TECHNICAL FIELD

The present invention relates to a technique for gradually generating chlorine dioxide gas.

### BACKGROUND ART

It is known that chlorine dioxide has strong oxidizability, and kills bacteria or degrades offensive odor components through its oxidizing action. Accordingly, chlorine dioxide is widely used as an antimicrobial agent, a deodorant, a fungicide, bleach, and the like. In these applications, chlorine dioxide is often used in the form of chlorine dioxide gas.

As an example of a chlorine dioxide gas generating method, a method in which an activator such as an organic acid or an inorganic acid is added to an aqueous chlorite solution is disclosed, for example, in JP 2005-29430A (Patent Document 1). In the method disclosed in Patent Document 1, the amount of chlorine dioxide gas generated is adjusted using a gas generation adjuster such as sepiolite or zeolite. Although not specifically described in Patent Document 1, it is assumed that, since sepiolite and zeolite are porous materials, the amount of gas generated is adjusted by retaining excessive gas in the gas generation adjuster when the amount of gas generated is large, and releasing the retained gas when the amount of gas generated is small.

However, it is not possible to sufficiently adjust the amount of gas generated merely through the physical adsorbing action, and it is not possible to sufficiently suppress an abrupt increase in the chlorine dioxide gas concentration after an activator is added to the aqueous chlorite solution. Accordingly, although Patent Document 1 states that chlorine dioxide gas is continuously generated, it will be appreciated that the effect is limited. Furthermore, the concentration of generated chlorine dioxide gas depends only on the concentration of chlorite, and control of the maximum concentration is not possible.

### PRIOR ART DOCUMENT

### Patent Document

Patent Document 1: JP 2005-29430A

### DISCLOSURE OF THE INVENTION

### Problem to be Solved by the Invention

There is demand for being able to freely control the concentration of generated chlorine dioxide gas and generate chlorine dioxide gas stably for a long period of time.

### Means for Solving Problem

The present invention is directed to a first chlorine dioxide gas generating method for generating chlorine dioxide gas at a stable concentration from a liquid composition, including obtaining the composition by mixing an aqueous chlorite solution, an activator that immediately adjusts a pH of the aqueous chlorite solution, thereby causing the aqueous chlorite solution to generate chlorine dioxide gas, and an activation inhibitor that slowly mitigates an action of the activator.

Note that, in a case in which the activation inhibitor is sodium silicate pentahydrate and an amount thereof added is 2% by weight or more with respect to an amount of the liquid composition excluding the activator, a case of further mixing 0.5% by weight or more of a catalyst for facilitating generation of chlorine dioxide gas within one minute after mixing the activator may be excluded (the same shall apply hereinafter).

The present invention is directed to a second chlorine dioxide gas generating method for generating chlorine dioxide gas at a stable concentration from a gel composition, including obtaining the composition by mixing an aqueous chlorite solution, an activator that immediately adjusts a pH of the aqueous chlorite solution, thereby causing the aqueous chlorite solution to generate chlorine dioxide gas, an activation inhibitor that slowly mitigates an action of the activator, and an absorbent resin.

The present invention is directed to a liquid composition for generating chlorine dioxide gas at a stable concentration, including an aqueous chlorite solution, an activator that immediately adjusts a pH of the aqueous chlorite solution, thereby causing the aqueous chlorite solution to generate chlorine dioxide gas, and an activation inhibitor that slowly mitigates an action of the activator.

The present invention is directed to a gel composition for generating chlorine dioxide gas at a stable concentration, including an aqueous chlorite solution, an activator that immediately adjusts a pH of the aqueous chlorite solution, thereby causing the aqueous chlorite solution to generate chlorine dioxide gas, an activation inhibitor that slowly mitigates an action of the activator, and an absorbent resin.

The present invention is directed to a first chlorine dioxide gas generating kit for generating chlorine dioxide gas at a stable concentration from a liquid composition, including:

a first agent containing an aqueous chlorite solution; and

a second agent containing an activator that immediately adjusts a pH of the aqueous chlorite solution, thereby causing the aqueous chlorite solution to generate chlorine dioxide gas, and an activation inhibitor that slowly mitigates an action of the activator,

wherein the composition is obtained by mixing the first agent and the second agent.

The present invention is directed to a second chlorine dioxide gas generating kit for generating chlorine dioxide gas at a stable concentration from a liquid composition, including:

a first agent containing an aqueous chlorite solution and an activation inhibitor; and

a second agent containing an activator that immediately adjusts a pH of the aqueous chlorite solution, thereby causing the aqueous chlorite solution to generate chlorine dioxide gas,

wherein the activation inhibitor slowly mitigates an action of the activator, and

the composition is obtained by mixing the first agent and the second agent.

The present invention is directed to a third chlorine dioxide gas generating kit for generating chlorine dioxide gas at a stable concentration from a gel composition, including:

a first agent containing an aqueous chlorite solution; and

a second agent containing an activator that immediately adjusts a pH of the aqueous chlorite solution, thereby causing the aqueous chlorite solution to generate chlorine dioxide gas, an activation inhibitor that slowly mitigates an action of the activator, and an absorbent resin,

wherein the composition is obtained by mixing the first agent and the second agent.

The present invention is directed to a fourth chlorine dioxide gas generating kit for generating chlorine dioxide gas at a stable concentration from a gel composition, including:

a first agent containing an aqueous chlorite solution and an activation inhibitor; and

a second agent containing an activator that immediately adjusts a pH of the aqueous chlorite solution, thereby causing the aqueous chlorite solution to generate chlorine dioxide gas, and an absorbent resin,

wherein the activation inhibitor slowly mitigates an action of the activator, and

the composition is obtained by mixing the first agent and the second agent.

With these configurations, when the components are mixed, the activator immediately acts, thereby causing chlorine dioxide gas to be immediately generated. Subsequently, the activation inhibitor slowly acts, thereby mitigating the action of the activator, and slowing down the generation of chlorine dioxide gas. Accordingly, an abrupt increase in the chlorine dioxide gas concentration in the early stage after mixing is inhibited, and chlorine dioxide gas is gradually released from the early stage. Accordingly, it is possible to generate chlorine dioxide gas stably for a long period of time. Furthermore, it is possible to freely control the concentration of generated chlorine dioxide gas by adjusting the amount of activation inhibitor added.

Hereinafter, preferred embodiments of the present invention will be described. Note that the scope of the present invention is not limited to the preferred embodiment examples described below.

In an aspect, it is preferable that the activation inhibitor is an alkali metal silicate or an alkaline-earth metal silicate.

With this configuration, when an alkali metal silicate or an alkaline-earth metal silicate is dissolved in an aqueous solution, hydroxide ions can be produced through hydrolysis. Thus, it is possible to slowly mitigate the action of an activator, which is typically an acid, through a neutralization reaction, and to freely control the concentration of chlorine dioxide gas.

In an aspect, it is preferable that the activation inhibitor is a sodium silicate.

With this configuration, it is possible to freely control the concentration of chlorine dioxide gas, at low cost, using a sodium silicate that is easily available and relatively inexpensive.

In an aspect, it is preferable that the activator is an inorganic acid or an organic acid, or a salt thereof, and

it is more preferable that the activator is an inorganic acid whose 1% aqueous solution has a pH of 1.7 or more and 2.4 or less, or a salt thereof,

the activator is an inorganic acid whose 1% aqueous solution has a pH of 3.8 or more and 4.5 or less, or a salt thereof, or

the activator is a mixture of an inorganic acid whose 1% aqueous solution has a pH of 1.7 or more and 2.4 or less, or a salt thereof, and an inorganic acid whose 1% aqueous solution has a pH of 3.8 or more and 4.5 or less, or a salt thereof.

With this configuration, it is possible to promptly and appropriately generate chlorine dioxide gas in the early stage after mixing the components.

In an aspect, it is preferable that the activator is sodium metaphosphate, or

the activator is sodium dihydrogen pyrophosphate.

With this configuration, it is possible to promptly and appropriately generate chlorine dioxide gas, at low cost, using sodium metaphosphate or sodium dihydrogen pyrophosphate that is easily available and stable.

In an aspect, it is preferable that the first agent and the second agent are respectively sealed in sealable containers.

With this configuration, it is possible to prevent oxygen or moisture in air from being mixed in, and to prevent the first agent and the second agent from deteriorating. Thus, it is possible to stably store the first agent and the second agent for a long period of time before use.

Further features and advantages of the technique according to the present invention will become apparent from the following description of illustrative and non-limiting embodiments with reference to the drawings.

### BRIEF DESCRIPTION OF THE DRAWINGS

FIG. 1 is a diagram illustrating the principle of a generating method for gradually releasing chlorine dioxide gas.

FIG. 2 is a graph showing the chlorine dioxide gas concentration in time series.

FIG. 3 is a schematic view showing the appearance of a chlorine dioxide gas generating kit.

FIG. 4 is a schematic view showing an aspect of a chlorine dioxide gas generating method.

FIG. 5 is a schematic view showing an example of a use mode of a gel composition.

### BEST MODE FOR CARRYING OUT THE INVENTION

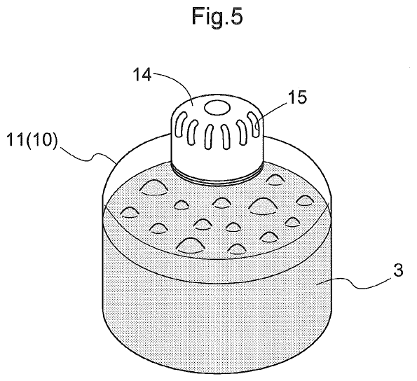

Hereinafter, a chlorine dioxide gas generating method, a liquid composition, a gel composition, and a chlorine dioxide gas generating kit according to an embodiment will be described. The chlorine dioxide gas generating method of this embodiment is a method for generating chlorine dioxide gas at a stable concentration, by mixing an aqueous chlorite solution, a fast-acting activator, a slow-acting activation inhibitor, and, optionally, an absorbent resin. In this embodiment, this method is performed using a chlorine dioxide gas generating kit K (see FIG. 3) including a first agent 1 containing an aqueous chlorite solution and a slow-acting activation inhibitor, and a second agent 2 containing a fast-acting activator, and, optionally, an absorbent resin. It is possible to generate chlorine dioxide gas at a stable concentration, from a liquid composition or a gel composition 3 (see FIG. 5) obtained by mixing the first agent 1 and the second agent 2 of the chlorine dioxide gas generating kit K.

In the description below, as an example, a case will be described in which chlorine dioxide gas is generated at a stable concentration from the gel composition 3 by also mixing the absorbent resin that is an optional component.

The aqueous chlorite solution is an aqueous solution containing chlorite. There is no particular limitation on the chlorite contained in the aqueous chlorite solution, as long as it is substantially stable, and is activated by being mixed with the activator and produces chlorine dioxide gas. Examples of the chlorite include alkali metal chlorite and alkaline-earth metal chlorite. Examples of the alkali metal chlorite include sodium chlorite (NaClO₂), potassium chlorite (KClO₂), and lithium chlorite (LiClO₂). Examples of the alkaline-earth metal chlorite include calcium chlorite (Ca (ClO₂)₂), magnesium chlorite (Mg (ClO₂)₂), and barium chlorite (Ba (ClO₂)₂). Of these, it is preferable to use sodium chlorite.

There is no particular limitation on the pH of the aqueous chlorite solution before mixing, but it is preferably 9 or more and 13 or less. The pH of the aqueous chlorite solution is more preferably 10 or more and 12.5 or less, and even more preferably 11 or more and 12 or less. If the pH is within this range, the chlorite in the aqueous chlorite solution can be stabilized and stably stored for a long period of time. The pH of the aqueous chlorite solution can be adjusted using an alkali agent. Examples of the alkali agent include sodium hydroxide (NaOH) and potassium hydroxide (KOH).

The activator activates the chlorite in the aqueous chlorite solution, when mixed with the solution, thereby causing the chlorite to generate chlorine dioxide gas. Examples of the activator include an inorganic acid and an organic acid, and a salt thereof. Examples of the inorganic acid include hydrochloric acid (HCl), carbonic acid (H₂CO₃), sulfuric acid (H₂SO₄), phosphoric acid (H₃PO₄), and boric acid (H₃BO₃). Examples of a salt of the inorganic acid include sodium hydrogen carbonate (NaHCO₃), sodium dihydrogen phosphate (NaH2PO4), and disodium hydrogen phosphate (Na₂HPO₄). As the inorganic acid and a salt thereof, it is also possible to use an anhydride (e.g., sulfuric anhydrite, pyrophosphoric acid, etc.), and, for example, it is preferable to use sodium dihydrogen pyrophosphate, or the like.

Examples of the organic acid include acetic acid (CH₃COOH), citric acid (H₃(C₃H₅O(COO)₃)), and malic acid (COOH(CHOH)CH₂COOH). Examples of a salt of the organic acid include sodium acetate (CH₃COONa), disodium citrate (Na₂H(C₃H₅O(COO)₃)), trisodium citrate (Na₃(C₃H₅O(COO)₃)), and disodium malate (COONa(CHOH)CH₂COONa).

The activator immediately adjusts the pH of the aqueous chlorite solution, when mixed with the aqueous chlorite solution. More specifically, the activator immediately lowers the pH of the aqueous chlorite solution, and provides an acidic atmosphere. In this sense, the activator can be said to be a “pH adjuster that immediately imparts acidity”. The activator adjusts the pH of the aqueous chlorite solution preferably to 2.5 or more and 6.8 or less.

The activator adjusts the pH of the aqueous chlorite solution more preferably to 3.5 or more and 6.5 or less, and even more preferably to 4.5 or more and 6.0 or less. Preferred examples of the activator include sodium metaphosphate whose 1% aqueous solution has a pH of 1.7 or more and 2.4 or less.

For example, if the chlorite contained in the aqueous chlorite solution is sodium chlorite, chlorous acid is produced following Formula (1) below, by adjusting the pH of the aqueous solution as described above to provide an acidic atmosphere.

NaClO₂+H⁺→Na⁺+HClO₂ (1)

Meanwhile, the equilibrium reaction in a case in which chlorine dioxide gas is dissolved in water is expressed by Formula (2) below.

2ClO₂+H₂O⇔HClO₂+HClO₃ (2)

At that time, Formula (3) below is obtained.

[HClO₂][HClO₃]/[ClO₂]=1.2×10⁻⁷ (3)

When chlorous acid is produced following Formula (1) by mixing the aqueous chlorite solution and the activator to set the aqueous chlorite solution to an acidic atmosphere, the equilibrium reaction shifts leftward in Formula (2) according to the theorem of Formula (3), and thus chlorine dioxide gas can be generated in the aqueous solution at an overwhelming probability.

In the chlorine dioxide gas generating method of this embodiment, in addition to the activator that immediately adjusts the pH of the aqueous chlorite solution (which will be referred to as a “first activator” in this example), a second activator that slowly adjusts the pH of the aqueous chlorite solution may be mixed as well. In this sense, the second activator can be said to be a “pH adjuster that slowly imparts acidity”.

The second activator may be an inorganic acid or organic acid with a level of acidity lower than that of the first activator, or a salt thereof. Preferred examples of the second activator include sodium pyrophosphate whose 1% aqueous solution has a pH of 3.8 or more and 4.5 or less.

The activation inhibitor slowly mitigates the action of the activator, when mixed with the aqueous chlorite solution together with the activator. The activation inhibitor slowly mitigates the action of the activator of immediately lowering the pH of the aqueous chlorite solution. The activation inhibitor may substantially be a material that slowly increases the pH of the aqueous chlorite solution. In this sense, the activation inhibitor can be said to be a “pH adjuster that slowly imparts alkalinity”. Examples of the activation inhibitor include an alkali metal silicate and an alkaline-earth metal silicate. Examples of the alkali metal silicate include a lithium silicate (mLi₂O.nSiO₂), a sodium silicate (mNa₂O.nSiO₂), and a potassium silicate (mK₂O.nSiO₂). Examples of the alkaline-earth metal silicate include a magnesium silicate (mMgO.nSiO₂), a calcium silicate (mCaO.nSiO₂), and a strontium silicate (mSrO.nSiO₂). Of these, it is preferable to use a sodium silicate (in particular, a sodium metasilicate).

There is no particular limitation on the molar ratio (the above-mentioned n/m) between an oxide of an alkali metal or an alkaline-earth metal silicate and a silicon dioxide, but it is preferably 0.9 or more and 1.2 or less.

For example, if the activation inhibitor is a sodium metasilicate, the sodium metasilicate dissociates (hydrolyzes) in the aqueous solution as in Formula (4) below.

Na₂O.SiO₂+2H₂O→2NaOH+H₂SiO₃ (4)

In this manner, sodium hydroxide (NaOH) produced after a short period of time has passed after mixing with the aqueous chlorite solution acts so as to partially neutralize the fast-acting activator (an acid in this example), thereby slowly mitigating the action of the activator. As a result, an abrupt increase in the chlorine dioxide gas concentration in the early stage after mixing is inhibited, and chlorine dioxide gas can be gradually released from the early stage.

Meanwhile, as in Formula (4), metasilicic acid (H₂SiO₃) is also produced in addition to sodium hydroxide. Metasilicic acid is produced after a short period of time has passed after mixing with the aqueous chlorite solution, and acts as an acid, and, in this sense, silicon dioxide (SiO₂) from which metasilicic acid is produced is an example of the “pH adjuster that slowly imparts acidity”. Sodium hydroxide and metasilicic acid produced later further react with each other as in Formula (5) below.

2NaOH+H₂SiO₃→Na₂O.SiO₂+2H₂O (5)

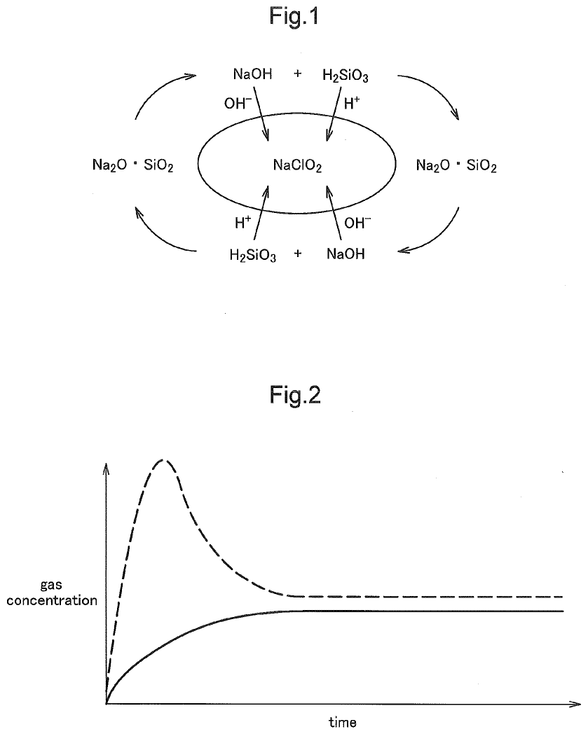

In this manner, sodium metasilicate serving as an activation inhibitor shifts between a state of being dissociated into sodium hydroxide and metasilicic acid and a state of being recombined, in the aqueous solution (see FIG. 1).

Then, sodium metasilicate in the state of being dissociated into sodium hydroxide and metasilicic acid slowly adjusts the pH of the aqueous chlorite solution. That is to say, in the state in which sodium metasilicate has dissociated into sodium hydroxide and metasilicic acid, metasilicic acid acts as a supply source of hydrogen ions (H⁺), and sodium hydroxide acts as a supply source of hydroxide ions (OH⁻), thereby slowly adjusting the pH of the aqueous chlorite solution. As a result, it is possible to slowly generate chlorine dioxide gas, and to generate chlorine dioxide gas at a stable concentration for a long period of time.

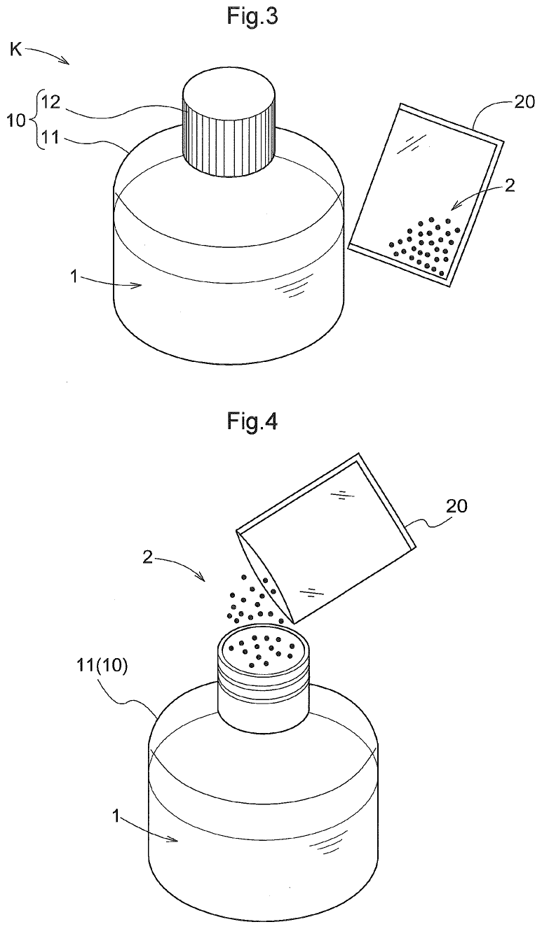

Note that, “generated at a stable concentration” means that, in a closed system, the concentration of generated chlorine dioxide gas slowly increases without having a peak in the early stage after mixing and then keeps a constant level (see FIG. 2), or, even if there is a peak, the ratio of the peak concentration relative to the final concentration is kept sufficiently low. In the case of the latter, the ratio of the peak concentration relative to the final concentration is, for example, preferably 1.3 or less, more preferably 1.2 or less, and even more preferably 1.1 or less.

Note that, in FIG. 2, a change in the concentration of chlorine dioxide gas when the activation inhibitor is mixed with the aqueous chlorite solution together with the activator in a closed system is indicated by the solid line, and a change in the concentration when the activation inhibitor is not mixed and only the activator is mixed is indicated by the broken line, for the sake of comparison.

Furthermore, according to the method of this embodiment, it is possible to freely control the concentration of generated chlorine dioxide gas.

Conventionally, the concentration of generated chlorine dioxide gas depends on the concentration of chlorite, and control of the maximum concentration was not possible, whereas, in this method, the maximum concentration (preferably, final concentration) of chlorine dioxide gas can be freely controlled by adjusting the amount of activation inhibitor added. Thus, it is possible to easily generate chlorine dioxide gas at a concentration suitable for the purpose of use.

The absorbent resin absorbs moisture, and forms a gel composition. Examples of the absorbent resin include a starch-based absorbent resin, a cellulose-based absorbent resin, and a synthetic polymer-based absorbent resin. Examples of the starch-based absorbent resin include a starch-acrylonitrile graft copolymer and a starch-acrylic acid graft copolymer. Examples of the cellulose-based absorbent resin include a cellulose-acrylonitrile graft copolymer and a cross-linked carboxymethylcellulose. Examples of the synthetic polymer-based absorbent resin include a polyvinyl alcohol-based absorbent resin and an acrylic-based absorbent resin.

The activator, the activation inhibitor, and the absorbent resin may be a solid (e.g., in a powdery form or a granular form) before mixed with the aqueous chlorite solution.

The chlorite concentration of the aqueous chlorite solution is preferably 0.01% by mass or more and 25% by mass or less, and more preferably 0.1% by mass or more and 15% by mass or less. Furthermore, the activator and the activation inhibitor may be contained, for example, in the following proportions, with respect to 1 L of 1% by mass aqueous chlorite solution. The activator is contained in a proportion of preferably 0.1% by mass or more and 3% by mass or less, and more preferably 0.2% by mass or more and 1.5% by mass or less. The activation inhibitor is contained in a proportion of preferably, 0.05% by mass or more and 30% by mass or less, and more preferably 0.5% by mass or more and 20% by mass or less, with respect to the mass of the activator.

The chlorine dioxide gas generating method of this embodiment may be performed using the chlorine dioxide gas generating kit K shown in FIG. 3. The chlorine dioxide gas generating kit K includes a first agent 1 containing an aqueous chlorite solution, and a second agent 2 containing a fast-acting activator, a slow-acting activation inhibitor, and an absorbent resin. In the chlorine dioxide gas generating kit K, the first agent 1 and the second agent 2 are respectively sealed in sealable containers. In this embodiment, the first agent 1 formed as a liquid (aqueous chlorite solution) is contained in a first container 10 mainly constituted by a container main body 11 made of plastic. The first container 10 has a sealing cap 12, and, when the sealing cap 12 is attached to the container main body 11 in a liquid-tight manner, the first agent 1 is sealed in the sealable first container 10.

Furthermore, the second agent 2 formed as a solid is contained in a second container 20 obtained by sticking plastic films to each other. The second container 20 may be obtained by stacking two plastic films and causing their entire peripheral edge portions to adhere to each other, or by folding one plastic film in half and causing the peripheral edge portions other than the folded portion to adhere to each other. In this manner, the second agent 2 is sealed in the sealable second container 20.

There is no limitation on the material and the shape of the first container 10 and the second container 20, as long as they are sealable containers. The material for forming the first container 10 and the second container 20 is not limited to plastic, and may be, for example, metal. Furthermore, the shape of the first container 10 is not limited to a fixed shape, and may be a deformable shape. The shape of the second container 20 is not limited to a deformable shape, and may be a fixed shape. Moreover, a configuration may also be employed in which the first agent 1 and the second agent 2 are contained in an integrated container having two container sections, and can be mixed with each other by bringing the two container sections into communication with each other at the time of use.

In the chlorine dioxide gas generating kit K of this embodiment, the first agent 1 is distributed in the form of an aqueous chlorite solution, and the storage safety is excellent. For example, the storage safety is higher than that in a case of distributing an aqueous chlorite solution in which chlorine dioxide gas is dissolved while keeping the pH acidic.

Chlorine dioxide gas can be actually generated using the chlorine dioxide gas generating kit K as follows. That is to say, as shown in FIG. 4, the sealing cap 12 is detached from the container main body 11 of the first container 10 containing the first agent 1. Furthermore, the second container 20 containing the second agent 2 is opened by cutting the plastic film. Then, when the second agent 2 in the second container 20 is inserted into the first container 10 (the container main body 11), the first agent 1 and the second agent 2 are mixed with each other. In this manner, the aqueous chlorite solution, the fast-acting activator, the slow-acting activation inhibitor, and the absorbent resin are mixed with each other in the first container 10 (the container main body 11).

Then, the content is converted into a gel form in the first container 10 (the container main body 11), and chlorine dioxide gas is generated at a stable concentration from the obtained gel composition 3 (see FIG. 5). If an opening cap 14 having a plurality of openings 15 is attached to the container main body 11, chlorine dioxide gas generated at a stable concentration is released via the openings 15 into a room. Thus, an antibacterial effect, a deodorant effect, and the like can be stably provided for a long period of time due to the strong oxidizability of chlorine dioxide gas gradually released at a stable concentration.

In the description above, a configuration may also be employed in which the second agent 2 does not contain the absorbent resin, and only the aqueous chlorite solution, the fast-acting activator, and the slow-acting activation inhibitor are mixed with each other. In this case, chlorine dioxide gas can be generated at a stable concentration from the obtained liquid composition. Also, in this case, an antibacterial effect, a deodorant effect, and the like can be provided stably for a long period of time due to the strong oxidizability of chlorine dioxide gas gradually released at a stable concentration.

Furthermore, in the description above, a configuration may also be employed in which the slow-acting activation inhibitor is contained not in the second agent 2 but in the first agent 1, and the aqueous chlorite solution and the slow-acting activation inhibitor are stored in the first container 10 and are mixed with the fast-acting activator (and the absorbent resin) at the time of use. Also in this case, chlorine dioxide gas can be generated at a stable concentration, and an antibacterial effect, a deodorant effect, and the like can be stably provided for a long period of time due to the strong oxidizability of chlorine dioxide gas gradually released at a stable concentration.

Hereinafter, the present invention will be described in more detail by way of examples.

### EXAMPLE 1

First, 17500 ppm of aqueous sodium chlorite solution was prepared by dissolving 7 g of sodium chlorite in 400 mL of pure water. Then, 10 g of 3% hydrochloric acid and 0.56 g of sodium dihydrogen phosphate serving as an activator, and 0.23 g of sodium silicate (Na₂O._{0.95}SiO₂) serving as an activation inhibitor were mixed with the aqueous sodium chlorite solution. Subsequently, the mixed liquid was stored in a sealed state at room temperature, and the pH of the mixed liquid and the concentration of generated chlorine dioxide gas were measured in a closed system.

### EXAMPLE 2

The pH of the mixed liquid and the concentration of chlorine dioxide gas were measured as in Example 1, except that the amount of sodium dihydrogen phosphate added as an activator was set to 1.17 g, and that the amount of sodium silicate added as an activation inhibitor was set to 0.33 g.

### EXAMPLE 3

The pH of the mixed liquid and the concentration of chlorine dioxide gas were measured as in Example 1, except that the amount of sodium dihydrogen phosphate added as an activator was set to 1.52 g, and that the amount of sodium silicate added as an activation inhibitor was set to 0.45 g.

### Comparative Example 1

The pH of the mixed liquid and the concentration of chlorine dioxide gas were measured as in Example 1, except that the amount of sodium dihydrogen phosphate added as an activator was set to 0.09 g, and that an activation inhibitor was not added.

Table 1 below shows the measurement results.

TABLE 1

| | | Elapsed time (days) | | | | | | |
|---|---|---|---|---|---|---|---|---|
| | | 0 | 1 | 2 | 9 | 18 | 24 | 31 |
| Ex. 1 | pH | 3.3 | 5.2 | 5.5 | 5.6 | 5.7 | 5.8 | 5.8 |
| | ClO₂ | 0 | 504 | 609 | 550 | 533 | 517 | 535 |
| | (ppm) | | | | | | | |
| Ex. 2 | pH | 3.6 | 5.2 | 5.4 | 5.6 | 5.6 | 5.7 | 5.9 |
| | ClO₂ | 0 | 468 | 448 | 439 | 475 | 458 | 444 |
| | (ppm) | | | | | | | |
| Ex. 3 | pH | 4.3 | 5.2 | 5.4 | 5.6 | 5.7 | — | 5.8 |
| | ClO₂ | 0 | 193 | 255 | 223 | 236 | — | 245 |
| | (ppm) | | | | | | | |
| Com. | pH | 3.0 | 5.3 | 5.7 | 5.8 | 5.9 | 5.9 | 5.9 |
| Ex. 1 | ClO₂ | 0 | 1397 | 887 | 779 | 736 | 684 | 656 |
| | (ppm) | | | | | | | |

It was seen that, in Comparative Example 1, the concentration of chlorine dioxide gas abruptly increased in the early stage after mixing and reached a peak, and then gradually decreased, whereas, in Examples 1 to 3, even when a strong acid was used as an activator, chlorine dioxide gas was gradually released.

### EXAMPLE 4

First, 11875 ppm of aqueous sodium chlorite solution was prepared by dissolving 4.75 g of sodium chlorite in 400 mL of pure water. Then, 9.3 g of 3% hydrochloric acid and 0.82 g of sodium dihydrogen phosphate serving as an activator, and 0.3 g of sodium silicate (Na₂O._{0.95}SiO₂) serving as an activation inhibitor were mixed with the aqueous sodium chlorite solution. Subsequently, the mixed liquid was stored in a sealed state at room temperature, and the pH of the mixed liquid and the concentration of generated chlorine dioxide gas were measured in a closed system. Furthermore, 9 days after mixing, the system was set to an accelerated environment, and the accelerated environment was maintained for 2 days. The accelerated environment was realized by increasing the temperature in the system to 54° C. and maintaining the temperature. Subsequently, the system was returned to that of a normal environment (i.e., the temperature was returned to room temperature), and then the pH of the mixed liquid and the concentration of generated chlorine dioxide gas were measured. Note that, due to the accelerated environment for 2 days, the state after 18 days substantially corresponds to that after 68 days in the normal environment (see Chinese Disinfection Technology Standards).

### Comparative Example 2

The pH of the mixed liquid and the concentration of chlorine dioxide gas were measured as in Example 4, except that an activation inhibitor was not added.

Table 2 below shows the measurement results.

TABLE 2

| | | Elapsed time (days) | | | | | |
|---|---|---|---|---|---|---|---|
| | | | | | | (Accelerated | |
| | | 0 | 1 | 2 | 4 | environment) | 18 |
| Ex. 4 | pH | 3.4 | 4.8 | 5.2 | 5.4 | (Stored at | 5.8 |
| | ClO₂ | 0 | 370 | 383 | 376 | 54° C.) | 370 |
| | (ppm) | | | | | | |
| Com. | pH | 2.9 | 4.4 | 4.9 | 5.9 | (Stored at | 6.2 |
| Ex. 2 | ClO₂ | 0 | 1077 | 1093 | 1047 | 54° C.) | 373 |
| | (ppm) | | | | | | |

It was seen that, in Comparative Example 2, the concentration of chlorine dioxide gas prominently decreased after long-term storage, whereas, in Example 4, chlorine dioxide gas was gradually released and the concentration thereof was maintained over a long period of time.

### EXAMPLE 5

Assumed as being a gel composition (gel agent), 113600 ppm of aqueous sodium chlorite solution was prepared by dissolving 45.44 g of sodium chlorite in 400 mL of pure water. Then, 25 g of sodium dihydrogen phosphate serving as an activator, and 1.33 g of sodium silicate (Na₂O._{0.95}SiO₂) serving as an activation inhibitor were mixed with the aqueous sodium chlorite solution. In this test, in order to simplify the pH measurement and the gas concentration measurement, the experiment was performed without mixing the absorbent resin. Subsequently, the mixed liquid assumed as being a gel composition was stored in a non-sealed state at room temperature, and the pH of the mixed liquid and the concentration of generated chlorine dioxide gas were measured in an open system.

### EXAMPLE 6

The pH of the mixed liquid and the concentration of chlorine dioxide gas were measured as in Example 5, except that the amount of sodium dihydrogen phosphate added as an activator was set to 31 g, and that the amount of sodium silicate added as an activation inhibitor was set to 2.67 g.

### EXAMPLE 7

The pH of the mixed liquid and the concentration of chlorine dioxide gas were measured as in Example 5, except that the amount of sodium dihydrogen phosphate added as an activator was set to 33 g, and that the amount of sodium silicate added as an activation inhibitor was set to 4 g.

### EXAMPLE 8

The pH of the mixed liquid and the concentration of chlorine dioxide gas were measured as in Example 5, except that the amount of sodium dihydrogen phosphate added as an activator was set to 45 g, and that the amount of sodium silicate added as an activation inhibitor was set to 5.34 g.

### Comparative Example 3

The pH of the mixed liquid and the concentration of chlorine dioxide gas were measured as in Example 5, except that the amount of sodium dihydrogen phosphate added as an activator was set to 20 g, and that an activation inhibitor was not added.

Table 3 below shows the measurement results.

TABLE 3

| | | Elapsed time (days) | | | | | | | |
|---|---|---|---|---|---|---|---|---|---|
| | | 0 | 1 | 4 | 10 | 16 | 32 | 39 | 41 |
| Ex. 5 | pH | 5.3 | 5.7 | 5.3 | 5.3 | 5.3 | 5.3 | 5.4 | 5.4 |
| | ClO₂ | 0 | 2220 | 2254 | 1719 | 1245 | 1107 | 915 | 754 |
| | (ppm) | | | | | | | | |
| Ex. 6 | pH | 5.4 | 5.7 | 5.5 | 5.4 | 5.4 | 5.5 | 5.5 | 5.5 |
| | ClO₂ | 0 | 2079 | 1473 | 1362 | 1332 | 1208 | 982 | 716 |
| | (ppm) | | | | | | | | |
| Ex. 7 | pH | 5.7 | 5.8 | 5.5 | 5.6 | 5.5 | 5.7 | 5.7 | 5.7 |
| | ClO₂ | 0 | 1601 | 1594 | 1480 | 1312 | 1029 | 861 | 639 |
| | (ppm) | | | | | | | | |
| Ex. 8 | pH | 5.6 | 5.7 | 5.5 | 5.5 | 5.5 | 5.5 | 5.5 | 5.6 |
| | ClO₂ | 0 | 1776 | 1820 | 1833 | 1712 | 1362 | 986 | 797 |
| | (ppm) | | | | | | | | |
| Com. | pH | 4.9 | 5.6 | 5.6 | 5.3 | 5.2 | 5.1 | 5.1 | 5.1 |
| Ex. 3 | ClO₂ | 0 | 3633 | 3263 | 1860 | 1719 | 696 | 619 | 558 |
| | (ppm) | | | | | | | | |

It was seen that, in the open system, the concentration of chlorine dioxide gas on the whole gradually decreased over time, but, in Examples 5 to 8, the level of decrease in the concentration of chlorine dioxide gas was kept small compared with that of Comparative Example 3.

In the description above, embodiments (including examples) of the chlorine dioxide gas generating method, the liquid composition, the gel composition, and the chlorine dioxide gas generating kit K were described in detail by way of specific examples, but the scope of the present invention is not limited to the foregoing specific examples and embodiments. The examples and embodiments disclosed in this specification are, in all respects, illustrative and not limiting. Various modifications may be made without departing from the gist of the invention.

### DESCRIPTION OF REFERENCE SIGNS

1 First agent

2 Second agent

3 Gel composition

10 First container (sealable container)

11 Container main body

12 Sealing cap

14 Opening cap

15 Opening

20 Second container (sealable container)

K Chlorine dioxide gas generating kit

1. A chlorine dioxide gas generating method for generating chlorine dioxide gas at a stable concentration from a liquid composition, comprising obtaining the composition by mixing an aqueous chlorite solution, an activator that immediately adjusts a pH of the aqueous chlorite solution, thereby causing the aqueous chlorite solution to generate chlorine dioxide gas, and an activation inhibitor that slowly mitigates an action of the activator
(excluding a case of further mixing 0.5% by weight or more of a catalyst for facilitating generation of chlorine dioxide gas within one minute after mixing the activator, in a case in which the activation inhibitor is sodium silicate pentahydrate and an amount thereof added is 2% by weight or more with respect to an amount of the liquid composition excluding the activator).

2. A chlorine dioxide gas generating method for generating chlorine dioxide gas at a stable concentration from a gel composition, comprising obtaining the composition by mixing an aqueous chlorite solution, an activator that immediately adjusts a pH of the aqueous chlorite solution, thereby causing the aqueous chlorite solution to generate chlorine dioxide gas, an activation inhibitor that slowly mitigates an action of the activator, and an absorbent resin.

3. A liquid composition for generating chlorine dioxide gas at a stable concentration, comprising an aqueous chlorite solution, an activator that immediately adjusts a pH of the aqueous chlorite solution, thereby causing the aqueous chlorite solution to generate chlorine dioxide gas, and an activation inhibitor that slowly mitigates an action of the activator
(excluding those further comprising 0.5% by weight or more of a catalyst for facilitating generation of chlorine dioxide gas within one minute after mixing the activator, in a case in which the activation inhibitor is sodium silicate pentahydrate and an amount thereof contained is 2% by weight or more with respect to an amount of the liquid composition excluding the activator).

4. A gel composition for generating chlorine dioxide gas at a stable concentration, comprising an aqueous chlorite solution, an activator that immediately adjusts a pH of the aqueous chlorite solution, thereby causing the aqueous chlorite solution to generate chlorine dioxide gas, an activation inhibitor that slowly mitigates an action of the activator, and an absorbent resin.

5. A chlorine dioxide gas generating kit for generating chlorine dioxide gas at a stable concentration from a liquid composition, comprising:
a first agent containing an aqueous chlorite solution; and
a second agent containing an activator that immediately adjusts a pH of the aqueous chlorite solution, thereby causing the aqueous chlorite solution to generate chlorine dioxide gas, and an activation inhibitor that slowly mitigates an action of the activator,
wherein the composition is obtained by mixing the first agent and the second agent.

6. A chlorine dioxide gas generating kit for generating chlorine dioxide gas at a stable concentration from a liquid composition, comprising:
a first agent containing an aqueous chlorite solution and an activation inhibitor; and
a second agent containing an activator that immediately adjusts a pH of the aqueous chlorite solution, thereby causing the aqueous chlorite solution to generate chlorine dioxide gas,
wherein the activation inhibitor slowly mitigates an action of the activator, and
the composition is obtained by mixing the first agent and the second agent
(excluding those in which at least one of the first agent and the second agent further contains 0.5% by weight or more of a catalyst for facilitating generation of chlorine dioxide gas within one minute after mixing the agents, in a case in which the activation inhibitor is sodium silicate pentahydrate and an amount thereof contained is 2% by weight or more with respect to an amount of the liquid composition excluding the activator).

7. A chlorine dioxide gas generating kit for generating chlorine dioxide gas at a stable concentration from a gel composition, comprising:
a first agent containing an aqueous chlorite solution; and
a second agent containing an activator that immediately adjusts a pH of the aqueous chlorite solution, thereby causing the aqueous chlorite solution to generate chlorine dioxide gas, an activation inhibitor that slowly mitigates an action of the activator, and an absorbent resin,
wherein the composition is obtained by mixing the first agent and the second agent.

8. A chlorine dioxide gas generating kit for generating chlorine dioxide gas at a stable concentration from a gel composition, comprising:
a first agent containing an aqueous chlorite solution and an activation inhibitor; and
a second agent containing an activator that immediately adjusts a pH of the aqueous chlorite solution, thereby causing the aqueous chlorite solution to generate chlorine dioxide gas, and an absorbent resin,
wherein the activation inhibitor slowly mitigates an action of the activator, and
the composition is obtained by mixing the first agent and the second agent.

9. The chlorine dioxide gas generating kit according to claim 5, wherein the activation inhibitor is an alkali metal silicate or an alkaline-earth metal silicate.

10. The chlorine dioxide gas generating kit according to claim 9, wherein the activation inhibitor is a sodium silicate.

11. The chlorine dioxide gas generating kit according to claim 5, wherein the activator is an inorganic acid or an organic acid, or a salt thereof.

12. The chlorine dioxide gas generating kit according to claim 11, wherein the activator is an inorganic acid whose 1% aqueous solution has a pH of 1.7 or more and 2.4 or less, or a salt thereof.

13. The chlorine dioxide gas generating kit according to claim 11, wherein the activator is sodium metaphosphate.

14. The chlorine dioxide gas generating kit according to claim 11, wherein the activator is an inorganic acid whose 1% aqueous solution has a pH of 3.8 or more and 4.5 or less, or a salt thereof.

15. The chlorine dioxide gas generating kit according to claim 11, wherein the activator is sodium dihydrogen pyrophosphate.

16. The chlorine dioxide gas generating kit according to claim 11, wherein the activator is a mixture of an inorganic acid whose 1% aqueous solution has a pH of 1.7 or more and 2.4 or less, or a salt thereof, and an inorganic acid whose 1% aqueous solution has a pH of 3.8 or more and 4.5 or less, or a salt thereof.

17. The chlorine dioxide gas generating kit according to claim 5, wherein the first agent and the second agent are respectively sealed in sealable containers.
